(19) United States
(12) Patent Application Publication
(10) Pub. No.: US 2010/0217247 A1
(43) Pub. Date: Aug. 26, 2010

(54) System and Methods for Minimizing Higher Order Aberrations Introduced During Refractive Surgery

(76) Inventors: Josef F. Bille, Heidelberg (DE); Frieder Loesel, Mannheim (DE); Luis Antonio Ruiz, Bogota (CO)

Correspondence Address: NYDEGGER & ASSOCIATES, 348 OLIVE STREET, SAN DIEGO, CA, 92103, US

(21) Appl. No.: 12/390,034

(22) Filed: Feb. 20, 2009

(51) Int. Cl. A61B 18/20

(52) U.S. Cl. 606/5

(57) ABSTRACT

A system and method are provided for minimizing the adverse effects of any optical aberrations, and particularly higher order aberrations, that may be introduced into an eye during the correction of a visual defect by photoablation (i.e. removal) of corneal tissue. In accordance with the present invention, after a predetermined time interval following the photoablation of tissue (e.g. about two weeks), the eye is evaluated for aberrations. Laser Induced Optical Breakdown (LIOB) is then performed on intrastromal tissue, as needed, to correct for the introduced aberrations.

### FIELD OF THE INVENTION

The present invention pertains generally to ophthalmic surgery. More particularly, the present invention pertains to systems and methods for correcting visual aberrations that are introduced into an eye during a laser surgery operation. The present invention is particularly, but not exclusively, useful as a system and method for using Laser Induced Optical Breakdown (LIOB) to weaken stromal tissue, and thereby correct visual aberrations that are introduced by the photoablation (removal) of corneal tissue during a surgical procedure.

### BACKGROUND OF THE INVENTION

Laser surgery that is performed on the cornea of an eye to correct a vision defect is typically accomplished in either of two different ways. For one, tissue can be removed from the cornea by photoablation to reshape the eye. Examples of such a surgical operation are the familiar PRK and LASIK procedures. Apart from photoablation, a more recently established surgical operation involves only the weakening of tissue rather than its removal. More specifically, this weakening of the cornea is done by cutting tissue in predetermined patterns inside the stroma of the cornea by a process known as Laser Induced Optical Breakdown (LIOB). The result is a redistribution of biomechanical stresses in the weakened stromal tissue that responds to intraocular pressure, to thereby reshape the cornea for correction of the vision defect.

Of the two different types of operations mentioned above, the removal of tissue by photoablation is able to accomplish more extensive corrections (i.e. provide greater diopter changes), than is possible with the second type operation wherein stromal tissue is only weakened by LIOB. Although photoablation may be required for the more extensive refraction corrections, or may otherwise be preferable, photoablation is known to sometimes introduce unwanted visual aberrations. More specifically, as an eye stabilizes after the surgical removal of tissue (i.e. photoablation), the eye can reshape in an unpredicted way that will introduce these unwanted aberrations.

Whenever corneal tissue is removed (i.e. photoablated) from the eye, it is essential that a sufficient amount of tissue remain. Obviously, there are limits to how much tissue can be actually removed. Further, the visual aberrations noted above, and particularly the higher order aberration, can be very detrimental or extremely annoying if left uncorrected. Unfortunately, it can happen that the introduced aberrations are not manifest until weeks after the initial corrective surgery.

In light of the above, it is an object of the present invention to provide a system and method for refining a laser surgical operation to correct for visual aberrations that may be introduced during a primary surgical operation. Still another object of the present invention is to provide a system and method for removing or minimizing visual aberrations, and particularly higher order aberrations, that may be introduced as an eye stabilizes after a surgical laser operation. Yet another object of the present invention is to provide a system and method for eliminating or minimizing surgically introduced visual aberrations that are simple to implement, are easy to use and are comparatively cost effective.

### SUMMARY OF THE INVENTION

In accordance with the present invention, an eye is evaluated after it has been subjected to a laser surgical operation for the correction of a vision defect. Specifically, the eye is evaluated after the operation to determine whether any visual aberrations were somehow inadvertently introduced into the eye during the surgical operation. If so, it is envisioned that the newly introduced aberrations will be corrected by selectively weakening stromal tissue. For the present invention this is to be done by the process of Laser Induced Optical Breakdown (LIOB). Further, as recognized by the present invention, this subsequent correction by LIOB may be most appropriate when the primary surgical operation has involved the removal of corneal tissue by photoablation (e.g. PRK or LASIK).

It often happens that visual aberrations, including higher order aberrations, are introduced into an eye as a result of a surgical procedure. Of particular interest here are the unpredicted aberrations that are caused by the removal (i.e. photoablation) of tissue during a primary surgical procedure. In some instances, these aberrations may be immediately detectable. On the other hand, as is more often the case, they do not fully manifest themselves until sometime after the initial surgery. This can be as much as two weeks, or more. In any event, it may either be undesirable or impossible to remove (photoablate) additional tissue to correct any aberrations that may have been surgically introduced. On the other hand, LIOB may still be possible as it essentially requires no additional removal of tissue. Furthermore, LIOB is known to be effective for correcting all orders of aberrations.

As envisioned for the present invention, a system for minimizing optical aberrations that may be introduced into an eye during laser surgery includes a first laser unit (e.g. an excimer laser). This first laser unit actually performs the laser surgery by ablating corneal tissue to achieve a predetermined refractive correction. After the laser surgery (photoablation) has been completed, the eye is then evaluated to detect any aberrations that may have been introduced into the eye by the first laser unit. If such aberrations are present, a second laser unit (e.g. a pulsed femtosecond laser) is used to cause Laser Induced Optical Breakdown (LIOB) on intrastromal tissue of the eye. The consequence here is a redistribution of biomechanical stresses in the stroma that, in response to intraocular pressure in the eye, will reshape the cornea to minimize the aberrations introduced by the earlier photoablation.

It is to be appreciated that the aberrations introduced by the first laser unit may be higher order aberrations. If so, the higher order aberrations are identified with reference to at least one offset axis, wherein the offset axis is substantially parallel to the visual axis of the eye. The second laser unit is then used to weaken stromal tissue by LIOB with reference to the offset axis to correct the higher order aberrations.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a cross section view of a cornea of an eye, positioned for laser surgery in accordance with the present invention;

FIG. 2 is a time-line presentation of functional tasks to be performed in accordance with the methodology of the present invention; and

FIG. 3 is a top plan view of exemplary LIOB patterns to be used for the correction of higher order aberrations introduced by photoablation during the laser surgery.

### DESCRIPTION OF THE PREFERRED EMBODIMENTS

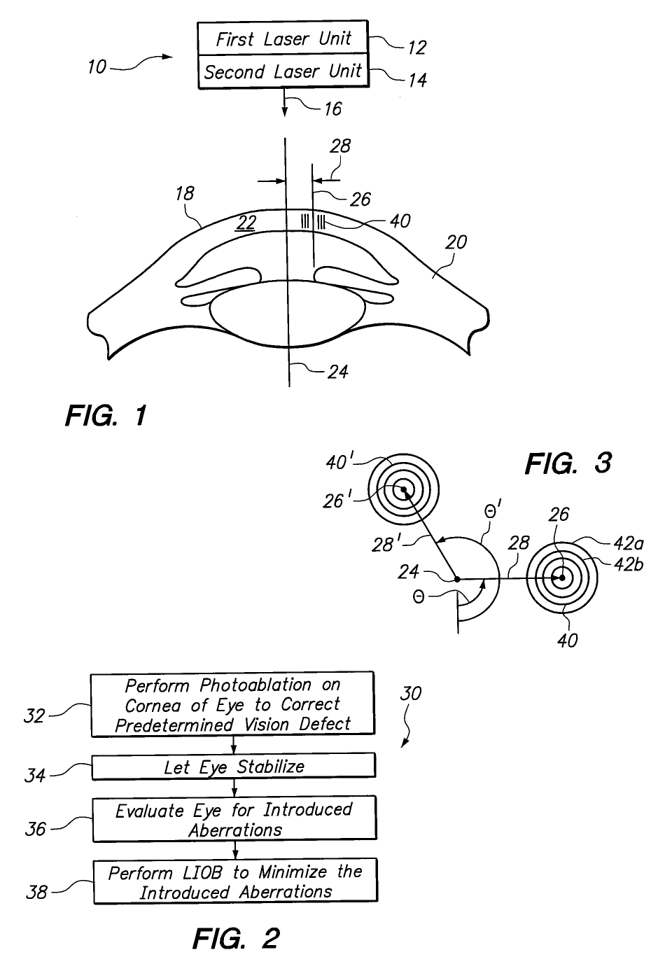

Referring initially to FIG. 1, a system in accordance with the present invention is shown and is generally designated 10. As shown, the system 10 includes a first laser unit 12 and a second laser unit 14. Further, FIG. 1 shows that the first laser unit 12 and the second laser unit 14 are both positioned to direct their respective laser beams along a substantially same laser beam path 16 toward the cornea 18 of an eye 20. Preferably, the first laser unit 12 includes an “excimer” type laser that is capable of photoablating tissue of the cornea 18. For example, it is envisioned for the present invention that the first laser unit 12 is capable of performing typical ophthalmic laser surgery, such the well-known PRK and LASIK procedures. Further, the second laser unit 14 is preferably a pulsed femtosecond type laser that is capable of reshaping the cornea 18 by weakening tissue in the stroma 22 of eye 20 through the process of Laser Induced Optical Breakdown (LIOB).

Still referring to FIG. 1, the eye 20 is shown to define a visual axis 24, and the beam path 16 from the system 10 is shown generally aligned with the visual axis 24. FIG. 1 also shows an exemplary offset axis 26 that is located at a radial distance 28 from the visual axis 24, and is substantially parallel to the visual axis 24. As will be more clearly appreciated with subsequent disclosure presented below, the actual location of the offset axis 26 and the magnitude of the radial distance 28 will depend on specific characteristics of higher order visual aberrations in the eye 20. In particular, the concern here is for aberrations that may have been introduced into the eye 20 during a photoablation of tissue in the cornea 18 by the first laser unit 12. A more systematic appreciation of when corrections for these aberrations (e.g. higher order aberrations) will be required can be made with reference to FIG. 2.

In FIG. 2, a time-line presentation of the methodology of the present invention is set forth and is generally designated 30. As shown, the functional tasks to be performed by the methodology 30 begin with a refractive correction of the cornea 18 (see block 32). Specifically, this refractive correction is made by the first laser unit 12 to correct a predetermined vision defect, and it will typically involve the photoablation of tissue (e.g. a PRK or LASIK procedure).

Block 34 indicates that the eye 20 may need some time to stabilize after the photoablation contemplated by block 32. Although no stabilization time may be required for some procedures, it can happen that as much as two weeks, or more, may be required for the results of photoablation to properly stabilize. In any event, as indicated by the block 36 in the methodology 30, after the eye 20 has stabilized, it is evaluated for aberrations that may have been introduced during the task shown in block 32. As envisioned for the present invention, and by way of example, this evaluation may detect higher order aberrations, such as astigmatism, coma or trefoil. Regardless of the type or order of these aberrations, they should be somehow minimized. As indicated in block 38 of the methodology 30, the present invention envisions minimizing introduced aberrations by performing LIOB on the cornea 18.

Although symmetrical and asymmetrical aberrations of different orders may be introduced during a primary laser procedure (block 32), block 38 indicates that LIOB as a corrective surgical procedure is appropriate in all cases. In particular, the present invention anticipates the higher order aberrations that require refractive corrections offset from the visual axis 24. With reference to FIG. 3, it is to be appreciated that such aberrations are to be treated by a pattern(s) 40 of LIOB cuts 42, where the pattern(s) 40 is (are) oriented on respective offset axes 26 and 26′. The patterns 40 and 40′ shown in FIG. 3 are exemplary.

As shown in FIG. 3, the patterns 40 and 40′ differ from each other according to their angular orientation (respectively shown in FIG. 3 by the angles θ and θ′). Further, they may also differ from each other in the magnitude of their respective radial distances 28 and 28′ from the visual axis 24. Despite these differences, each pattern 40 and 40′ will include a series of cuts 42 (the cylindrical cuts 42a and 42b for pattern 40 are only exemplary). For purposes of the present invention, the pattern(s) 40 result from LIOB performed on tissue in the stroma 22 that weakens this tissue. Intraocular pressure will then cause the weakened tissue to reshape the cornea 18 for correction of the introduced aberrations.

While the particular System and Methods for Minimizing Higher Order Aberrations Introduced During Refractive Surgery as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for correcting aberrations introduced during refractive surgery on an eye which comprises the steps of:
removing corneal tissue to correct a visual defect of the eye;
evaluating visual aberrations introduced into the eye during the removing step; and
weakening stromal tissue to correct the introduced visual aberrations.

2. A method as recited in claim 1 wherein the removing step is accomplished by photoablating tissue.

3. A method as recited in claim 2 wherein the photoablation of tissue is accomplished by a procedure selected from a group comprising LASIK and PRK.

4. A method as recited in claim 1 wherein the weakening step is accomplished by Laser Induced Optical Breakdown (LIOB).

5. A method as recited in claim 1 wherein the evaluating step further comprises the steps of:
identifying the visual aberrations; and
quantifying the visual aberrations to establish parameters for the weakening step.

6. A method as recited in claim 1 wherein the aberration is a higher order aberration.

7. A method as recited in claim 6 wherein the eye defines a visual axis and the method further comprises the steps of:
identifying at least one offset axis wherein the offset axis is substantially parallel to the visual axis; and
performing the weakening step with reference to the offset axis to correct the higher order aberrations.

8. A method as recited in claim 1 wherein the removing step is accomplished using an excimer laser.

9. A method as recited in claim 1 wherein the weakening step is accomplished using a pulsed femtosecond laser.

10. A method as recited in claim 1 wherein the evaluating step and the weakening step are performed subsequent to the removing step, and after a time interval following the removing step greater than approximately two weeks.

11. A method for minimizing optical aberrations introduced into an eye during laser surgery which comprises the steps of:
ablating corneal tissue to achieve a predetermined refractive correction;
evaluating the eye, subsequent to the ablating step, to detect any aberrations introduced into the eye during the ablating step; and
causing Laser Induced Optical Breakdown (LIOB) on intrastromal tissue of the eye for a redistribution of biomechanical stresses in the stroma to minimize the introduced aberrations.

12. A method as recited in claim 11 wherein the ablating step is accomplished using an excimer laser in a procedure selected from a group comprising LASIK and PRK.

13. A method as recited in claim 11 wherein the causing step is accomplished using a pulsed femtosecond laser.

14. A method as recited in claim 11 wherein the introduced aberrations are higher order aberrations, wherein the eye defines a visual axis, and wherein the method further comprises the steps of:
identifying at least one offset axis wherein the offset axis is substantially parallel to the visual axis; and
performing the causing step with reference to the offset axis to correct the higher order aberrations.

15. A method as recited in claim 11 wherein the evaluating step and the causing step are accomplished subsequent to the ablating step, and after a time interval following the ablating step greater than approximately two weeks.

16. A system for minimizing optical aberrations introduced into an eye during laser surgery which comprises:
a first laser unit for ablating corneal tissue to achieve a predetermined refractive correction;
a means for evaluating the eye to detect any aberrations introduced into the eye by the first laser unit; and
a second laser unit for causing Laser Induced Optical Breakdown (LIOB) on intrastromal tissue of the eye with a consequent redistribution of biomechanical stresses in the stroma to minimize the introduced aberrations.

17. A system as recited in claim 16 wherein the first laser unit is an excimer laser.

18. A system as recited in claim 16 wherein the second laser unit is a pulsed femtosecond laser.

19. A system as recited in claim 16 wherein the aberrations introduced by the first laser unit are higher order aberrations.

20. A system as recited in claim 19 wherein the eye defines a visual axis and the higher order aberrations are identified with reference to at least one offset axis, wherein the offset axis is substantially parallel to the visual axis, and wherein the second laser unit is used to weaken stromal tissue by LIOB with reference to the offset axis to correct the higher order aberrations.
